(19) United States
(12) Patent Application Publication
(10) Pub. No.: US 2022/0281933 A1
(43) Pub. Date: Sep. 8, 2022

(54) FUSION TOXIN PROTEINS FOR TREATMENT OF DISEASES RELATED TO CMV INFECTIONS

(71) Applicants: KØBENHAVNS UNIVERSITET, Copenhagen K (DK); DANMARKS TEKNISKE UNIVERSITET, Lyngby (DK)

(72) Inventors: Thomas Nitschke KLEDAL, Lyngby (DK); Mette Marie ROSENKILDE, Hellerup (DK)

(21) Appl. No.: 17/626,675

(22) PCT Filed: Jun. 25, 2020
(86) PCT No.: PCT/EP2020/067785

§ 371 Date: Jan. 12, 2022

(30) Foreign Application Priority Data
Jul. 12, 2019 (EP) 19186000.6

(51) Int. Cl. C07K 14/52; C07K 14/21; A61K 47/64; A61P 31/12

(52) U.S. Cl. CPC C07K 14/521; C07K 14/21; A61K 47/642; A61P 31/12; A61K 38/00

(57) ABSTRACT

The present invention relates to immunotoxins useful in treating diseases related to CMV infection. The invention also relates to use of the immunotoxin and pharmaceutical compositions comprising the immunotoxin as a medicament, and a kit for treatment or prevention of CMV infection comprising the immunotoxin.

### TECHNICAL FIELD OF THE INVENTION

The present invention relates to immunotoxins useful in treating diseases related to CMV infection. The invention also relates to use of the immunotoxin and pharmaceutical compositions comprising the immunotoxin as a medicament, and a kit for treatment or prevention of CMV infection comprising the immunotoxin.

### BACKGROUND OF THE INVENTION

Cytomegalovirus

Cytomegalovirus (CMV) is an important human pathogen and a major opportunist, which emerges to cause disease in immuno-compromised subjects, such as AIDS patients, neonates, and individuals who have been given immunosuppressive drugs e.g. as part of a transplantation regimen. In these individuals, the consequences of CMV in re-emerging and/or acute infections can be dire, including retinitis, encephalitis, and pneumocystis, among other pathologies. Furthermore, in immuno-competent hosts, CMV establishes a persistent lifelong infection through which it has been linked to a variety of inflammatory conditions including coronary artery occlusion following heart transplant and atherectomy and restenosis following angioplasty. CMV interacts with leukocytes during acute infection of the host as well as during lifelong latency. As such, leukocytes are important players in CMV-induced diseases and have been implicated in the acute phase of infection as vehicles for dissemination of virus and as sites of residence during lifelong latency.

Currently no cure for CMV infection is available. Viral suppressants have been used to inhibit CMV replication, but they carry strong side effects and serve only to inhibit infection. The most common drugs for the treatment of CMV infection in transplantation patients and HIV/AIDS patients are the generic drugs Ganciclovir and Acyclovir, originally developed for herpes simplex virus (HSV). Ganciclovir and Acyclovir have a suppressing effect on CMV as well as on HSV. Foscavir has also been used to suppress CMV infection, but has been shown to cause intolerable nausea. Prevymis, a small molecule terminase inhibiter also inhibits viral replication. Letermovir inhibits the CMV DNA terminase complex (pUL51, pUL56, and pUL89), which is required for viral DNA processing and packaging.

In conclusion, none of the existing drugs can eradicate the infection, but merely halts the CMV disease progression in immuno-compromised or immuno-suppressed patients. Thus, there is room for improvement both in efficacy and in toxicity levels.

Immunotoxins

An immunotoxin is a ligand combined with a toxin, which can be used to kill cells expressing receptors for the ligand. Immunotoxin treatment is also known as ligand-targeted therapeutics. Thus, the immunotoxins contain a targeting moiety (a ligand) for delivery and a toxic moiety for cytotoxicity. The ligands currently used are monoclonal antibodies, cytokines/growth factors and soluble receptors. An advantage with immunotoxins over e.g. traditional chemotherapy drugs is, that the cells need not be dividing to be killed. Furthermore, if the immunotoxin is efficiently and selectively internalized, side effects will not occur in antigen negative cells.

In general, however, immunotoxins have not shown impressive levels of efficacy. A common problem is that they are not sufficiently specific for the diseased cells, and furthermore, often are incapable of efficiently entering the diseased cells to exert its cytotoxic effects. Immunotoxins also result in higher levels of systemic toxicity than other therapies, presumably because of non-specific uptake of the immunotoxin.

Currently, new approaches to immunotoxins are being explored to overcome problems of toxicity, immunogenicity, and heterogeneity of antigen expression. These approaches include the use of genetic engineering to fuse the translocation and catalytic domains of toxins to human single chain antibodies and to use phage display to select high affinity, tumour-selective ligands. Use of bivalent constructs can also increase the affinity and potency. Other approaches, centres around the selection of ligands that target tumour vascular endothelium and the targeting of oncogene products or differentiation antigens. In spite of that research on immunotoxins has been ongoing in the last three decades, no immunotoxin against virus related diseases is available on the market.

Immunotoxin Internalization

Most immunotoxins are developed against cells that have undergone malignant transformation and as a part of this transformation therefore overexpress a certain antigen or a group of certain antigens. Even though these antigens are over expressed on the transformed cells, they are rarely specific for the transformed cells, but are often also expressed on normal cells. Thus, only a few cellular antigens are over expressed on transformed cells. Therefore, to avoid undesired toxicity by killing normal cells expressing the target antigen, drug developers are restricted to target very few candidate disease antigens, and drug developers have therefore traditionally been restricted to select a target antigen solely based on the cell type distribution. Consequently, many immunotoxins have not been able to efficiently enter the target cells, even though they bind the target antigen with high affinity, resulting in inadequate potency.

Immunotoxins for Treatment of Diseases Related to CMV Infections

Prior attempts to treat diseases related to CMV infections using immunotoxin strategies have not been successful.

Both toxins linked to polyclonal and monoclonal antibodies have been utilized for targeting CMV-infected cells. However, antibodies are not easy to develop against GPCRs and they will in general not recognize GPCRs with variability in the recognition site. Thus, antibody binding will be highly susceptible to receptor variations not associated with receptor functionality, thereby increasing the risk of escape variants (development of resistance). Consequently, strategies based on antibodies have not produced convincing in vivo data, possibly due to insufficient (selective) targeting and/or internalization of the immunotoxins.

Alternatively, the targeting moiety of the immunotoxin may be a peptide instead of an antibody. In WO 2008/003327, CMV-infected cells were targeted using an immunotoxin comprising a peptide designed for targeting constitutively internalizing CMV encoded receptors. One of the receptors of choice was the CMV-specific receptor named US28, which is a G protein coupled receptor encoded by human cytomegalovirus open reading frame US28. A challenge for design of an efficient immunotoxin targeting the US28 receptor is the presence of the human homologous receptor named CX3CR1. Thus, selectivity for US28 over CX3CR1 is a key characteristic for obtaining a safe immunotoxin with minimal off-target issues. In WO 2008/003327, a mutated version of the chemokine CX3CL1 (fractalkine), i.e. the natural ligand of CX3CR1, was used as targeting moiety. Using this strategy, selectivity for US28 over CX3CR1 was achieved.

Because of the presence of the off-target human homologous receptor, CX3CR1, it is not sufficient to develop an immunotoxin with high affinity and potency against cells expressing US28. Instead, the immunotoxin should have (i) high specificity, i.e. high affinity for US28 expressing cells compared to CX3CR1 expressing cells in a competitive binding environment and (ii) high killing specificity, i.e. high potency against US28 expressing cells compared to CX3CR1 expressing cells. To advance an immunotoxin into clinic it is important to enhance US28 selectivity and killing specificity in order to limit adverse side effects and reduce production costs.

Hence, an improved immunotoxin would be advantageous, and in particular, a safe immunotoxin with high selectivity and killing specificity for US28 expressing cells over CX3CR1 expressing cells would be advantageous.

### SUMMARY OF THE INVENTION

By designing immunotoxins with high affinity towards a constitutively internalizing CMV encoded receptor, efficient uptake of the immunotoxin by the infected cell, and thereby the death of the infected cell is accomplished. Since the internalization of the immunotoxin is considered the rate-limiting step in immunotoxin-mediated cytotoxicity, targeting a constitutively internalizing receptor will solve a central problem in use of immunotoxin based drugs. Moreover, the immunotoxins presented herein are designed to discriminate between healthy cells and infected cells.

Thus, an object of the present invention relates to immunotoxins that target a constitutively internalizing receptor, ensuring that the immunotoxin will be transported into the target cell, where it can exert its function, i.e. kill the cell. Furthermore, the immunotoxins of the invention target with high accuracy only CMV-infected cells and can be used in the treatment or prevention of CMV-infection or CMV-associated diseases.

In particular, it is an object of the present invention to provide an immunotoxin with improved selectivity and killing specificity that may be used as a safe drug with high efficacy.

Thus, one aspect of the invention relates to an immunotoxin comprising:
i) a targeting moiety comprising an amino acid sequence selected from:
   a) SEQ ID NO:1, or
   b) an amino acid sequence having at least 80% sequence identity to SEQ ID NO:1, and
ii) a toxin,

wherein the amino acid residue in position 49 is replaced by an alanine (A) residue and the amino acid residues in positions 1-6 are replaced with the amino acid sequence ILDNGVS in the N-terminal end.

Another aspect of the present invention relates to a pharmaceutical composition comprising an immunotoxin according to the present invention or a pharmaceutically acceptable salt thereof.

Yet another aspect of the present invention is to provide an immunotoxin or a pharmaceutical composition according to the present invention for use as a medicament.

Still another aspect of the present invention is to provide an immunotoxin or a pharmaceutical composition according to the present invention for use in the treatment or prevention of CMV infections or CMV-associated diseases.

An even further aspect of the present invention is to provide a kit comprising:
i) an immunotoxin or a pharmaceutical composition according to the present invention,
ii) one or more additional therapeutic agents, and
iii) optionally, instructions for use,

wherein i) and ii) are for simultaneous, separate or sequential administration.

Another aspect of the present invention is to provide a nucleic acid sequence comprising a sequence encoding an immunotoxin according to the present invention.

A further aspect of the present invention is to provide a recombinant expression vector comprising a nucleotide sequence according to the present invention operably linked to one or more control sequences suitable for directing the production of the immunotoxin in a suitable host.

Still another aspect of the present invention is to provide a recombinant host cell comprising an expression vector according to the present invention.

An even further aspect of the present invention relates to a method of producing the immunotoxin according to the present invention comprising the steps of:
a) providing a host cell according to the present invention,
b) cultivating said host cell under conditions suitable for the expression of said immunotoxin; and
c) isolating said immunotoxin.

### BRIEF DESCRIPTION OF THE FIGURES

FIG. 1 shows competition binding experiments in stable inducible clones of US28-expressing HEK293 cells (circles) using ¹²⁵1-CCL2 as radioligand comparing SYN001 (black symbols) and SYN004 (white symbols).
FIG. 2 shows competition binding experiments in stable inducible clones of CX3CR1-expressing HEK293 cells (squares) using ¹²⁵1-CX3CL1 as radioligand comparing SYN001 (black symbols) and SYN004 (white symbols).

FIG. 3 shows competition binding experiments in stable inducible clones of US28-expressing HEK293 cells (circles) using ¹²⁵I-CCL2 as radioligand comparing SYN000 (black symbols) and SYN003 (white symbols).

FIG. 4 shows competition binding experiments in stable inducible clones of CX3CR1-expressing HEK293 cells (squares) using ¹²⁵I-CX3CL1 as radioligand comparing SYN000 (black symbols) and SYN003 (white symbols).

FIG. 5 shows killing experiments comparing SYN001 (black symbols) and SYN004 (white symbols) in stable inducible clones of US28-expressing HEK293 cells (circles) and CX3CR1-expressing HEK293 cells (squares).

FIG. 6 shows killing experiments comparing SYN000 (black symbols) and SYN003 (white symbols) in stable inducible clones of US28-expressing HEK293 cells (circles) and CX3CR1-expressing HEK293 cells (squares).

FIG. 7 shows a schematic diagram of the domain structure of; (i) human CX3CL1 (SS=signal sequence, CX3CL1=chemokine domain, Stalk=Mucin-like stalk, M=Membrane spanning part and C=cytoplasmic domain), and (ii) Pseudomonas aeruginosa Exotoxin A (SS=signal sequence, Domain I=receptor binding domain, Domain II=translocation domain, Ib=Domain Ib with unknown function and Domain III=enzymatically active domain). The amino acid numbering for the precursor protein is given above each protein. Disulphide bridges are indicated below each protein with a square bracket along with numbering of the amino acids involved. Furin cleaves between amino acids 304 and 305 of domain II of Exotoxin A.

FIG. 8 shows a schematic diagram of the drug substance candidates. A mutation in a given domain is written in single letter code of the amino acid involved along with its number corresponding to the amino acid position in the native protein (i.e. human CX3CL1 or Pseudomonas aeruginosa Exotoxin A) on which the immunotoxin is based. E.g. C312S means that cysteine at position number 312 has been substituted with serine. Single letter code is also used at the N- and C-terminus of the constructs and between domains. A dashed line between two domains indicates that the amino acids are connected.

The present invention will now be described in more detail in the following.

### DETAILED DESCRIPTION OF THE INVENTION

Definitions

Prior to discussing the present invention in further details, the following terms and conventions will first be defined:

Immunotoxin

In the present context, the term “immunotoxin” refers to a bifunctional molecule comprising a targeting moiety for delivery (a ligand) and a toxic moiety (toxin) for cytotoxicity. The immunotoxin can be used to kill cells expressing receptors for the ligand.

Immunotoxins may also be referred to as fusion toxin proteins (FTP). Thus, the terms “immunotoxin” and “fusion toxin protein (FTP)” are used interchangeably herein.

Ligand or Targeting Moiety

In the present context, the term “ligand” refers to any amino acid, peptide, polypeptide or protein, which possesses a specific binding affinity to a receptor or an antigen, e.g. originating from a virus.

Herein the ligand is used to specifically target the immunotoxin to a desired location. Thus, the ligand is also referred to as a “targeting moiety”. Consequently, the terms “ligand” and “targeting moiety” are used interchangeably herein.

The targeting moiety of the immunotoxins described herein is preferably in the form of a peptide or polypeptide.

Peptide or Polypeptide

In the present context, the terms “peptide” or “polypeptide” refers to a polymer composed of amino acid residues, related naturally occurring structural variants, and synthetic non-naturally occurring analogs thereof linked via peptide bonds. The terms “peptide” and “polypeptide” are used interchangeably herein.

Polypeptides may be produced recombinantly or synthetically. Recombinant production of polypeptides may be accomplished by introducing expression vectors comprising nucleic acid encoding the polypeptide of interest in known expression systems, as would be known to the person skilled in the art. Synthetic polypeptides can be synthesized, for example, using an automated polypeptide synthesizer.

Conventional notation is used herein to portray polypeptide sequences: the left-hand end of a polypeptide sequence is the amino-terminus (N-terminus); the right-hand end of a polypeptide sequence is the carboxyl-terminus (C-terminus).

Toxin

In the present context, the term “toxin” refers to any substance, being a protein or non-peptide, which is cytotoxic or cytostatic or that induce apoptosis or necrosis or that directly inhibits the replication, growth or dissemination of the pathogen, or that makes the infected cell vulnerable to the infected host immune response.

Examples of toxins include, but are not limited to, exotoxins, endotoxins, enzymatic toxins, pore-forming toxins, superantigens and ribosome inactivating protein (RIP).

Examples of enzymatic toxins include, but are not limited to, Pseudomonas exotoxin A, cholera toxin, diphtheria toxin, pertussis toxin, shiga toxin, botulinum toxin, tetanus toxin, anthrax toxin and staphylococcus aureus exfoliatin B.

Examples of pore-forming toxins include, but are not limited to, hemolysin, listeriolysin, anthrax EF, alpha toxin, pneumolysin, streptolysin O, leukocidin and perfringiolysin O.

Examples of ribosome inactivating proteins (RIPs) include, but are not limited to Pseudomonas exotoxin A, gelonin, bouganin, saporin, ricin, ricin A chain, bryodin, diphtheria, restrictocin and diphtheria toxin.

CX3CL1 and CX3CR1

In the present context, the term “CX3CL1” refers to a chemokine, which is a member of the CX3C chemokine family. Chemokines are low molecular weight proteins that regulates cell migration. Many chemokines also possess the capability to induce maturation, activation, proliferation, and differentiation of cells of the immune system. CX3CL1 is also known as fractalkine and neurotactin and the terms “CX3CL1”, “fractalkine” and “neurotactin” are therefore used interchangeably herein.

CX3CL1 comprises a chemokine domain, which is responsible for interaction with the corresponding chemokine receptor. The chemokine domain is defined by the amino acid sequence listed as SEQ ID NO:1. This amino acid sequence corresponds to positions 25 to position 99 of the native human CX3CL1 as given in the UniProt database under entry P78423.

CX3CL1 may be modified (i.e. mutated by deletion, insertion, and/or substitution, conjugated, etc.) in accordance with the present invention.

CX3CL1 exert its function through interaction with the corresponding receptor termed “CX3CR1”. Thus, in the present context, the term “CX3CR1” refers to the fractalkine receptor and has the amino acid sequence listed as SEQ ID NO:4. This amino acid sequence corresponds to the native human fractalkine receptor as given in the UniProt database under entry P49238.

Internalization

In the present context, the term “internalization” refers to transfer of an entity from the extracellular environment to the intracellular compartment of a cell. Thus, internalization of the immunotoxin as described herein refers to the entry of the immunotoxin into the intracellular compartments of a target cell, such as a cell expressing an antigen interacting with the targeting moiety of the immunotoxin.

Internalization of the immunotoxin may be mediated through a constitutively internalizing receptor, such as US28. Constitutively internalization refers to any antigen that is expressed at the plasma membrane and, without prior stimulation, is internalized into the cell cytoplasm or an intracellular compartment from the cell plasma membrane. The antigen internalization may be modulated by a ligand, and the internalized antigen may recycle to the plasma membrane or may be degraded after internalization.

US28

In the present context, the term “US28” refers to a G protein coupled receptor encoded by human cytomegalovirus open reading frame US28. US28 is a constitutively internalizing receptor and therefore chemokines or other compounds that binds US28 are internalized into cells expressing the receptor. In the present context, US28 has the amino acid sequence listed as SEQ ID NO:3. This amino acid sequence corresponds to the CX3CR1 homologue as given in the UniProt database under entry Q9IP69. However, US28 exist in different variants and the targeting of the immunotoxin as described herein is not limited to one specific variant, i.e. amino acid sequence, of US28.

Affinity

In the present context, the term “affinity” refers to binding affinity, i.e. the strength of the binding interaction between the immunotoxin and a receptor. The affinity is measured and reported as the equilibrium dissociation constant (Kᵢ) in a heterologous binding experiment. The smaller the Kᵢ value, the greater the binding affinity of the ligand for its target. The larger the Kᵢ value, the more weakly the target molecule and ligand are attracted to and bind to one another.

The Kᵢ may be measured by methods including, but not limited to, isothermal titration calorimetry (ITC), ELISA, gel-shift assays, pull-down assays, equilibrium dialysis, analytical ultracentrifugation, SPR, and spectroscopic assays, saturation binding experiments, and homologous and heterologous competition binding experiments.

Selectivity

In the present context, the term “selectivity” refers to the affinity of the immunotoxin for US28 versus the affinity of the immunotoxin for CX3CR1. This may be represented by the equation Kᵢ (US28)/Kᵢ (CX3CR1). The index of the dissociation constants is denoted “i” since this is a case of heterologous binding.

Potency

In the present context, the term “potency” refers to the reduction in cell viability upon administration of the immunotoxin. The potency is therefore defined as an inhibitory potency and is quantified by an IC50 value. Therefore, the potency against cells expressing US28 is herein denoted IC50(US28), whereas potency against cells expressing CX3CR1 is denoted IC50(CX3CR1). Typically, the IC50 value is reported in nM, with lower concentrations of immunotoxin corresponding to high potency and higher concentrations of immunotoxin corresponding to low potency.

Killing Specificity

In the present context, the term “killing specificity” refers to the ability of the immunotoxin to specifically kill cells expressing US28 over cells expressing CX3CR1. Killing specificity is quantified as the ratio between the (inhibitory) potency against cells expressing CX3CR1 and the (inhibitory) potency against cells expressing US28. Thus, the killing specificity may be calculated as IC50(CX3CR1)/IC50(US28), with high values indicating high killing specificity towards US28 expressing cells.

The killing specificity as defined herein is also in some instances referred to as the selectivity index. Consequently, the terms “killing specificity” and “selectivity index” are used interchangeably herein.

Cytomegalovirus (CMV) In the present context, the term “cytomegalovirus” or “CMV” refers to a virus in the family Herpesviridae. Human CMV (HCMV) also named “human herpesvirus 5” or “HHV-5” refers to a CMV that is capable of infecting humans.

CMV Infections and CMV-Associated Diseases

In the present context, the term “CMV infection” or “CMV-associated disease” refers to diseases, syndromes, maladies or mortality that are caused or associated with the presence of CMV in the diseased individual or evident from serological investigations of the diseased individual.

CMV is causing acute as well as chronic diseases. The acute diseases, which most often are associated with a high level of viral replication and characterized by affecting multiple organs are mononucleosis like syndromes, perinatal infections in premature infants, CMV syndrome in allograft recipients and disseminated infections in immuno-compromised patients, such as AIDS patients. The chronic infections, which most often is associated with a low level of viral replication are congenital infections, vascular diseases in transplant patients, vascular diseases in the normal host and inflammatory diseases, especially in the gastrointestinal tract.

Furthermore, the presence of CMV as determined by either molecular or serological methods is associated with increased morbidity and mortality in transplant recipients. Indeed, prophylaxis of CMV has been shown to decrease all-cause mortality post transplantation. Also, CMV infection is associated with organ rejection in solid organ transplant recipients and with graft versus host disease in haematopoietic stem cell recipients.

Amino Acid Sequence

In the present context, the term “amino acid sequence” refers to a series of consecutive amino acids comprising naturally occurring amino acids and/or artificial amino acid analogues. An amino acid sequence may be a polymer of amino acids, such as a protein, polypeptide, peptide, etc. Given the degeneracy of the genetic code, one or more nucleic acids, or the complementary nucleic acids thereof, that encode a specific polypeptide sequence can be determined from the polypeptide sequence.

Variants

In the present context, the term “variant” refers to a polypeptide comprising a sequence, which differs (by deletion of an amino acid, insertion of an amino acid, and/or substitution of an amino acid for a different amino acid) in one or more amino acid positions from that of a native polypeptide sequence. The variant sequence may be a non-naturally occurring sequence, i.e. a sequence not found in nature. Preferably, a “variant” retains the same function as the native polypeptide. Thus, “variants” result in immunotoxins that have the same or enhanced affinity, selectivity, potency or killing specificity as the native polypeptide.

Fragments

In the present context, the term “fragment” refers to a polypeptide comprising a sequence, which is an excerpt of a larger parent polypeptide. Thus, the fragment sequence shares a stretch of consecutive amino acids with the parent sequence, but is of a reduced length. Preferably, a “fragment” retains the same function as the parent polypeptide. Thus, “fragments” result in immunotoxins that have the same or enhanced affinity, selectivity, potency or killing specificity as the native polypeptide.

Pharmaceutical Composition

In the present context, the term “pharmaceutical composition” refers to a composition suitable for pharmaceutical use in an individual. A pharmaceutical composition generally comprises an effective amount of an active agent, such as an immunotoxin, and a carrier including, but not limited to, a pharmaceutically acceptable carrier.

Effective Amount

In the present context, the term “effective amount” refers to a dosage or amount sufficient to produce a desired effect. The desired effect may comprise an objective or subjective improvement in the recipient of the dosage or amount, such as prophylactic or therapeutic treatment of an individual.

Prophylactic/Preventive Treatment

In the present context, the term “prophylactic treatment” refers to a treatment administered to an individual who does not display signs or symptoms of a disease, pathology, or medical disorder, or displays only early signs or symptoms of a disease, pathology, or disorder, such that treatment is administered for the purpose of diminishing, preventing, or decreasing the risk of developing the disease, pathology, or medical disorder. A prophylactic treatment functions as a preventative treatment against a disease or disorder, and therefore the terms “prophylactic treatment” and “preventive treatment” are used interchangeably herein.

Therapeutic Treatment

In the present context, the term “therapeutic treatment” refers to a treatment administered to an individual who displays symptoms or signs of pathology, disease, or disorder, in which treatment is administered to the individual for the purpose of diminishing or eliminating those signs or symptoms of pathology, disease, or disorder.

Nucleotide Acid Sequence

In the present context, the term “nucleotide acid sequence” (e.g. a nucleic acid, polynucleotide, oligonucleotide, etc.) refers to a polymer of nucleotides comprising nucleotides A,C,T,U,G, or other naturally occurring nucleotides or artificial nucleotide analogues. Either the given nucleic acid or the complementary nucleic acid can be determined from any specified polynucleotide sequence. Unless otherwise indicated, a particular nucleic acid sequence also implicitly encompasses conservatively modified variants thereof (e.g. degenerate codon substitutions) and complementary sequences and as well as the sequence explicitly indicated. Specifically, degenerate codon substitutions may be achieved by generating sequences in which the third position of one or more selected (or all) codons is substituted with mixed-base and/or deoxyinosine residues. In the present context, the term “nucleic acid” is used interchangeably with “gene”, “cDNA”, and “mRNA encoded by a gene”.

Nucleic Acid Derived from a Gene

In the present context, the phrase “nucleic acid derived from a gene” refers to a nucleic acid for whose synthesis the gene, or a subsequence thereof, has ultimately served as a template. Thus, an mRNA, a cDNA reverse transcribed from an mRNA, an RNA transcribed from that cDNA, a DNA amplified from the cDNA, an RNA transcribed from the amplified DNA, etc., are all derived from the gene.

Protein targets of the immunotoxin may in some embodiments be described by their genetic origin (i.e. nucleic acid sequence) instead of their amino acid sequence.

Operably Linked

In the present context, the term “operably linked” refers to the covalent joining of two or more nucleotide sequences, by means of enzymatic ligation or otherwise, in a configuration relative to one another such that the normal function of the sequences can be performed. For example, the nucleotide sequence encoding a pre-sequence or secretory leader is operably linked to a nucleotide sequence for a polypeptide if it is expressed as a pre-protein that participates in the secretion of the polypeptide: a promoter or enhancer is operably linked to a coding sequence if it affects the transcription of the sequence; a ribosome binding site is operably linked to a coding sequence if it is positioned so as to facilitate translation. Generally, “operably linked” means that the nucleotide sequences being linked are contiguous and, in the case of a secretory leader, contiguous and in reading phase. Linking is accomplished by ligation at convenient restriction sites. If such sites do not exist, then synthetic oligonucleotide adaptors or linkers are used, in conjunction with standard recombinant DNA methods.

Control Sequences

In the present context, the term “control sequences” refers to sequences that include all components, which are necessary or advantageous for the expression of a polypeptide of the present invention. Each control sequence may be native or foreign to the nucleotide sequence encoding the polypeptide. Such control sequences include, but are not limited to, a leader, polyadenylation sequence, pro-peptide sequence, promoter, signal peptide sequence, and transcription terminator. Typically, the control sequences include at least a promoter, and transcriptional and translational stop signals. The control sequences may be provided with linkers for the purpose of introducing specific restriction sites facilitating ligation of the control sequences with the coding region of the nucleotide sequence encoding a polypeptide.

Expression Vector

In the present context, the term “expression vector” refers to a DNA molecule, linear or circular, that comprises a segment encoding a polypeptide, and which is operably linked to additional segments that provide for its transcription.

Host Cell

In the present context, the term “host cell” refers to any cell type, which is susceptible to transformation with a nucleic acid construct. The host cell may be eukaryotic or prokaryotic.

Recombinant

In the present context, the term “recombinant” refers to a cell, virus, nucleotide, or vector that has been modified by the introduction of a heterologous (or foreign) nucleic acid or the alteration of a native nucleic acid, or that the protein or polypeptide has been modified by the introduction of a heterologous amino acid, or that the cell is derived from a cell so modified. Recombinant cells express nucleic acid sequences (e.g. genes) that are not found in the native (non-recombinant) form of the cell or express native nucleic acid sequences (e.g. genes) that would be abnormally expressed under-expressed, or not expressed at all.

Sequence Identity

In the present context, the term “sequence identity” is here defined as the sequence identity between genes or proteins at the nucleotide, base or amino acid level, respectively. Specifically, a DNA and a RNA sequence are considered identical if the transcript of the DNA sequence can be transcribed to the identical RNA sequence.

Thus, in the present context “sequence identity” is a measure of identity between proteins at the amino acid level and a measure of identity between nucleic acids at nucleotide level. The protein sequence identity may be determined by comparing the amino acid sequence in a given position in each sequence when the sequences are aligned. Similarly, the nucleic acid sequence identity may be determined by comparing the nucleotide sequence in a given position in each sequence when the sequences are aligned.

To determine the percent identity of two amino acid sequences or of two nucleic acids, the sequences are aligned for optimal comparison purposes (e.g., gaps may be introduced in the sequence of a first amino acid or nucleic acid sequence for optimal alignment with a second amino or nucleic acid sequence). The amino acid residues or nucleotides at corresponding amino acid positions or nucleotide positions are then compared. When a position in the first sequence is occupied by the same amino acid residue or nucleotide as the corresponding position in the second sequence, then the molecules are identical at that position. The percent identity between the two sequences is a function of the number of identical positions shared by the sequences (i.e., % identity=# of identical positions/total # of positions (e.g., overlapping positions)×100). In one embodiment, the two sequences are the same length.

In another embodiment, the two sequences are of different length and gaps are seen as different positions. One may manually align the sequences and count the number of identical amino acids. Alternatively, alignment of two sequences for the determination of percent identity may be accomplished using a mathematical algorithm. Such an algorithm is incorporated into the NBLAST and XBLAST programs of (Altschul et al. 1990). BLAST nucleotide searches may be performed with the NBLAST program, score=100, word length=12, to obtain nucleotide sequences homologous to a nucleic acid molecules of the invention. BLAST protein searches may be performed with the XBLAST program, score=50, word length=3 to obtain amino acid sequences homologous to a protein molecule of the invention.

To obtain gapped alignments for comparison purposes, Gapped BLAST may be utilized. Alternatively, PSI-Blast may be used to perform an iterated search, which detects distant relationships between molecules. When utilizing the NBLAST, XBLAST, and Gapped BLAST programs, the default parameters of the respective programs may be used. See http://www.ncbi.nlm.nih.gov. Alternatively, sequence identity may be calculated after the sequences have been aligned e.g. by the BLAST program in the EMBL database (www.ncbi.nlm.gov/cgi-bin/BLAST). Generally, the default settings with respect to e.g. “scoring matrix” and “gap penalty” may be used for alignment. In the context of the present invention, the BLASTN and PSI BLAST default settings may be advantageous.

The percent identity between two sequences may be determined using techniques similar to those described above, with or without allowing gaps. In calculating percent identity, only exact matches are counted. An embodiment of the present invention thus relates to sequences of the present invention that has some degree of sequence variation.

Substantially Homologous/Identical

In the present context, the term “substantially homologous” or “substantially identical” in the context of two nucleic acids or polypeptides, generally refers to two or more sequences or subsequences that have at least 40%, 60%, 80%, 90%, 95%, 96%, 97%, 98% or 99% nucleotide or amino acid residue identity, when compared and aligned for maximum correspondence, as measured using a comparison algorithm or by visual inspection.

Fusion Toxin Proteins

Cytomegalovirus (CMV) is a clinically important opportunistic viral pathogen in individuals with immature or compromised immune function. All approved drug therapies against human CMV (HCMV) used for prophylactic, pre-emptive, or treatment of HCMV infection or disease, targets the viral replication machinery. Although this strategy has been effective in some settings, the current approved nucleoside analogous drugs fail in preventing HCMV disease in other settings, e.g., lung-, heart-lung-, pancreas-, and allogeneic hematopoietic stem cell transplantation. Prevymis, a small molecule terminase inhibiter also inhibits viral replication. Letermovir inhibits the CMV DNA terminase complex (pUL51, pUL56, and pUL89) which is required for viral DNA processing and packaging. Prevymis prophylaxis inhibits CMV infection and disease in haematopoietic stem cell recipients. Importantly, the approved nucleoside analogous drugs have treatment-limiting side effects, including serious nephro-, neuro-, and hematologic toxicity, and are susceptible to the frequent development of drug-resistant strains, with single mutations commonly conferring resistance to multiple drugs across the class. While Prevymis is generally considered safe to use, Prevymis prophylaxis is still associated with high frequency (approximately 30%) break-through reactivation. Together, these limitations highlight the need for better strategies based on novel mechanisms of action to improve or complement existing therapies and to treat disease refractory to DNA polymerase inhibitors because of resistance.

Herein is presented a HCMV antiviral strategy based on targeting of HCMV-infected cells through their expression of a virus-encoded seven-transmembrane (7TM) chemokine receptor, US28. The HCMV antiviral strategy is effected by immunotoxins that are chimeric molecules comprising a toxin and a targeting moiety.

Upon infection of a cell by CMV, US28 is expressed on the surface of the infected cell and becomes capable of responding to chemokines in the environment. The US28 receptor binds a variety of human, murine, and virus-encoded CC chemokines. Interestingly, the CX3C chemokine, fractalkine (also termed CX3CL1), binds with a very high affinity to US28, while targeting only one additional receptor, namely, its cognate receptor, CX3CR1, thus decreasing the potential for unwanted off-target effects of a CX3CL1-based immunotoxin strategy.

Furthermore, the majority of the US28 receptors are localized within endosomes, away from the cell surface. This distribution is the result of rapid, constitutive, ligand-independent receptor internalization. Thus, chemokines or other compounds that binds US28 are internalized into the cell that express the receptor. Without being bound by theory, it is contemplated that the immunotoxins described herein benefit from this characteristic and upon binding to US28 are transported into the CMV infected cell, where the toxin can exert its cytotoxic function.

Because of the presence of the off-target human homologous receptor, CX3CR1, it is important that the immunotoxin has high affinity and potency for US28 expressing cells compared to CX3CR1 expressing cells. Herein, are presented immunotoxins, wherein the targeting moiety for US28 is a peptide based on the natural ligand fractalkine, but with a modified and improved amino acid sequence that result in a potent immunotoxin for treatment or prevention of CMV infections or CMV-associated diseases.

Thus, an aspect of the present invention relates to an immunotoxin comprising:
i) a targeting moiety comprising an amino acid sequence selected from:
   a) SEQ ID NO:1, or
   b) an amino acid sequence having at least 80% sequence identity to SEQ ID NO:1, and
ii) a toxin,

wherein the amino acid residue in position 49 is replaced by an alanine (A) residue and the amino acid residues in positions 1-6 are replaced with the amino acid sequence ILDNGVS in the N-terminal end.

The natural ligand of US28, CX3CL1 (fractalkine), consists of a chemokine domain, a mucin stalk, and a transmembrane domain which anchors CX3CL1 to the cell membrane. The chemokine domain of CX3CL1 has high affinity for US28. Herein, the chemokine domain is defined by the amino acid sequence listed as SEQ ID NO:1, corresponding to positions 25 to position 99 of the native human CX3CL1. The core mutations to SEQ ID NO:1 resulted in surprisingly potent immunotoxins. Without being bound by theory, it is believed that the phenylalanine to alanine substitution and the modified N-terminal end of the targeting moiety (ILDNGVS, SEQ ID NO:13), are causing the improved immunotoxins as described herein. Therefore, many different immunotoxins with similar functional characteristics can be envisioned by altering non-critical single amino acids or non-critical regions of amino acids of the targeting moiety. Thus, an embodiment of the present invention relates to the immunotoxin as described herein, wherein the amino acid sequence of (b) has at least 90% sequence identity to SEQ ID NO:1, such as at least 95%, such as at least 96%, such as at least 97%, such as at least 98%, such as at least 99% sequence identity to SEQ ID NO:1. Another embodiment of the present invention relates to the immunotoxin as described herein, wherein the N-terminal sequence of the targeting moiety is not QHHGVT (SEQ ID NO:14).

In other instances, it may be preferred that the targeting moiety closely resembles the chemokine domain of fractalkine, being only modified with the core mutations. Thus, an embodiment of the present invention relates to the immunotoxin as described herein, wherein the targeting moiety is SEQ ID NO:1 in which the amino acid residue in position 49 is replaced by an alanine (A) residue and the amino acid residues in positions 1-6 are replaced with the amino acid sequence ILDNGVS in the N-terminal end. This targeting moiety, comprising the two core mutations, is also represented by SEQ ID NO:2. Thus, a preferred embodiment of the present invention relates to the immunotoxin as described herein, wherein the targeting moiety comprises SEQ ID NO:2.

The immunotoxins may be produced recombinantly or synthetically. In the case of recombinant production, translation starts with the methionine that is bound to the initiator tRNA. Thus, an embodiment of the present invention relates to the immunotoxin as described herein, wherein the amino acid of said targeting moiety further comprises a methionine (M) residue as the first amino acid from the N-terminal end. An immunotoxin comprising a targeting moiety with an initiator methionine (M) is represented by SEQ ID NO:11. Therefore, an embodiment of the present invention relates to the immunotoxin as described herein, wherein the targeting moiety comprises SEQ ID NO:11.

The immunotoxins as described herein exert their cytotoxic effect specifically to HCMV infected cells by targeting of the US28 receptor. Thus, an embodiment of the present invention relates to the immunotoxin as described herein, wherein the targeting moiety binds to the US28 receptor. Another embodiment of the present invention relates to the immunotoxin as described herein, wherein the immunotoxin is internalized subsequent to binding US28.

The US28 receptor is a G protein coupled receptor encoded by human cytomegalovirus open reading frame US28. In the present context, the US28 receptor has the amino acid sequence represented by SEQ ID NO:3. However, US28 exist in different variants and the targeting of the immunotoxin as described herein is not limited to one specific variant but extends also to substantially homologous or substantially identical variants.

Thus, an embodiment of the present invention relates to the immunotoxin as described herein, wherein the US28 receptor comprises an amino acid sequence selected from:
i) SEQ ID NO:3, or
ii) an amino acid sequence having at least 80% sequence identity to SEQ ID NO:3.

Another embodiment of the present invention relates to the immunotoxin as described herein, wherein the US28 receptor comprises an amino acid sequence selected from:
i) SEQ ID NO:3, or
ii) an amino acid sequence having at least 90% sequence identity to SEQ ID NO:3, such as at least 95%, such as at least 96%, such as at least 97%, such as at least 98%, such as at least 99% sequence identity to SEQ ID NO:3.

The US28 receptor may also be defined at the genetic level. Therefore, in the present context, the US28 receptor is encoded by a nucleic acid sequence represented by SEQ ID NO:12. As the US28 receptor exist in different variants and the genetic code is subject to degeneracy, the US28 receptor as defined by its genetic code is not limited to a one single nucleic acid, but extend also to substantially homologous or substantially identical variants.

Therefore, an embodiment of the present invention relates to the immunotoxin as described herein, wherein the US28 receptor is encoded by a nucleic acid sequence selected from:
i) SEQ ID NO:12, or
ii) a nucleic acid sequence having at least 80% sequence identity to SEQ ID NO:12.

Another embodiment of the present invention relates to the immunotoxin as described herein, wherein the US28 receptor is encoded by a nucleic acid sequence selected from:
i) SEQ ID NO:12, or
ii) a nucleic acid sequence having at least 90% sequence identity to SEQ ID NO:12, such as at least 95%, such as at least 96%, such as at least 97%, such as at least 98%, such as at least 99% sequence identity to SEQ ID NO:12.

The immunotoxins as described herein are designed for binding with strong affinity (Kᵢ) for US28. Thus, an embodiment of the present invention relates to the immunotoxin as described herein, wherein the immunotoxin binds US28 with a Kᵢ of 10⁻⁷ M or less, such as 10⁻⁸ M or less, such as 10⁻⁹ M or less, such as 10⁻¹⁹ M or less, such as 10⁻¹¹ M or less. The binding affinity is assayed in competition with ¹²⁸I-CCL2 or ¹²⁸I-CX3CL1 as radioligand. The exact assay is described further in the examples.

Since the human homologous receptor, CX3CR1, represents an undesirable off-target, it is advantageous if the immunotoxins have relatively low affinity for CX3CR1. This will not only increase the efficacy of the immunotoxins, but also reduce adverse effects as healthy cells are less affected by administration of the immunotoxin. Thus, an embodiment of the present invention relates to the immunotoxin as described herein, wherein the affinity of the immunotoxin for the human homologous receptor CX3CR1 is reduced as compared to the affinity of CX3CL1 (SEQ ID NO:1) for CX3CR1, such as at least 100-fold, such as at least 150-fold, such as 200-fold, or such as at least 250-fold reduced. Another embodiment of the present invention relates to the immunotoxin as described herein, wherein the immunotoxin binds the CX3CR1 with a Kᵢ of 10⁻⁶ or more. A further embodiment of the present invention relates to the immunotoxin as described herein, wherein the immunotoxin has increased affinity for US28 as compared to the affinity for CX3CR1, such as at least 75-fold, such as at least 100-fold, such as at least 150-fold, such as 200-fold, or such as at least 250-fold increased affinity.

Without being bound by theory, it is presumed that it is mainly the targeting moiety of the immunotoxins that are responsible for binding to the US28 receptor. Therefore, an embodiment of the present invention relates to the immunotoxin as described herein, wherein the targeting moiety binds US28 with a Kᵢ of 10⁻⁷ M or less, such as 10⁻⁸ M or less, such as 10⁻⁹ M or less, such as 10⁻¹⁹ M or less, such as 10⁻¹¹ M or less. Another embodiment of the present invention relates to the immunotoxin as described herein, wherein the affinity of the targeting moiety for the human homologous receptor CX3CR1 is reduced as compared to the affinity of CX3CL1 (SEQ ID NO:1) for CX3CR1, such as at least 100-fold, such as at least 150-fold, such as 200-fold, or such as at least 250-fold reduced. A further embodiment of the present invention relates to the immunotoxin as described herein, wherein the targeting moiety binds the CX3CR1 with a Kᵢ of 10⁻⁶ or more. Yet another embodiment of the present invention relates to the immunotoxin as described herein, wherein the targeting moiety has increased affinity for US28 as compared to the affinity for CX3CR1, such as at least 75-fold, such as at least 100-fold, such as at least 150-fold, such as 200-fold, or such as at least 250-fold increased affinity.

The human homologous receptor, CX3CR1, may in the present context be represented by the amino acid sequence according to SEQ ID NO:4. However, CX3CR1 as described herein is not limited to one specific variant, but extend also to substantially homologous or substantially identical variants. Therefore, an embodiment of the present invention relates to the immunotoxin as described herein, wherein the CX3CR1 receptor comprises an amino acid sequence according to SEQ ID NO:4.

Another embodiment of the present invention relates to the immunotoxin as described herein, wherein the CX3CR1 receptor comprises an amino acid sequence selected from:
i) SEQ ID NO:4, or
ii) an amino acid sequence having at least 80% sequence identity to SEQ ID NO:4, such as at least 90%, such as at least 95%, such as at least 96%, such as at least 97%, such as at least 98%, such as at least 99% sequence identity to SEQ ID NO:3.

Without being bound by theory, the toxin of the immunotoxin does not contribute in binding to and internalization of the immunotoxin into the CMV infected cells. Thus, the immunotoxins are functional utilizing a wide range of toxins. Therefore, an embodiment of the present invention relates to the immunotoxin as described herein, wherein the toxin is selected from one or more of the group consisting of exotoxins, endotoxins, enzymatic toxins, pore-forming toxins, superantigens and ribosome inactivating protein (RIP). Another embodiment of the present invention relates to the immunotoxin as described herein, wherein the toxin is a ribosome inactivating protein (RIP). A further embodiment of the present invention relates to the immunotoxin as described herein, wherein the toxin is selected from one or more of the group consisting of Pseudomonas exotoxin A, gelonin, bouganin, saporin, ricin, ricin A chain, bryodin, diphtheria, restrictocin, diphtheria toxin, and fragments or variants thereof.

Pseudomonas Exotoxin A is a very potent toxin capable of killing cells via its adenosine diphosphate-ribosylation domain that modifies elongation factor 2, leading to the arrest of protein synthesis and the initiation of apoptosis. Thus, an embodiment of the present invention relates to the immunotoxin as described herein, wherein the toxin is Pseudomonas exotoxin A (SEQ ID NO:5) or a fragment thereof.

Pseudomonas Exotoxin A comprises domains associated with translocation (domain II) and cytotoxicity (domains Ib and III), respectively. Therefore, an embodiment of the present invention relates to the immunotoxin as described herein, wherein the toxin comprises one or more fragments selected from the group consisting of the binding domain (domain II, SEQ ID NO:6), intermediate domain (domain Ib, SEQ ID NO:7) and the ADP-ribosylating domain (domain III, SEQ ID NO:8) of Pseudomonas exotoxin A. A preferred embodiment of the present invention relates to the immunotoxin as described herein, wherein the toxin comprises the binding domain (domain II, SEQ ID NO:6) and the ADP-ribosylating domain (domain III, SEQ ID NO:8) of Pseudomonas exotoxin A.

In a variant of Pseudomonas Exotoxin A, the C-terminal end is modified to increase cytotoxicity. Thus, an embodiment of the present invention relates to the immunotoxin as described herein, wherein the C-terminal amino acid sequence REDLK (SEQ ID NO:15) of the ADP-ribosylating domain is replaced by the amino acid sequence KDEL (SEQ ID NO:16). A preferred embodiment of the present invention relates to the immunotoxin as described herein, wherein the toxin is PE38KDEL (SEQ ID NO:9).

Another preferred embodiment of the present invention relates to the immunotoxin as described herein, wherein the immunotoxin comprises SEQ ID NO:9 and SEQ ID NO:2. An immunotoxin comprising the combination of (i) the targeting moiety being SEQ ID NO:1 in which the amino acid residue in position 49 is replaced by an alanine (A) residue and the amino acid residues in positions 1-6 are replaced with the amino acid sequence ILDNGVS in the N-terminal end, and (ii) the toxin being PE38KDEL, is represented by SEQ ID NO:10. Thus, a further preferred embodiment of the present invention relates to the immunotoxin as described herein, wherein the immunotoxin comprises SEQ ID NO:10.

As demonstrated in the examples herein, the immunotoxins described herein display surprisingly high potency towards US28 expressing cells as compared to CX3CR1 expressing cells. Therefore, an embodiment of the present invention relates to the immunotoxin as described herein, wherein the immunotoxin has increased potency against cell expressing US28 as compared to the potency against cells expressing CX3CR1, such as at least 150-fold, such as 175-fold, or such as at least 200-fold, such as at least 225-fold, such as at least 250-fold increased potency.

The immunotoxins as described herein are intended for medical use and may therefore form part of a pharmaceutical composition. For the purpose of medical use, the immunotoxin may be formulated as a pharmaceutically acceptable salt. Pharmaceutical compositions may comprise elements that are standard for medical use and would be known to the person skilled in the art. Thus, an aspect of the present invention relates to a pharmaceutical composition comprising an immunotoxin as described herein or a pharmaceutically acceptable salt thereof. Another embodiment of the present invention relates to the pharmaceutical composition as described herein, wherein the composition comprises a pharmaceutically acceptable carrier, diluent and/or excipient.

An aspect of the present invention relates to an immunotoxin as described herein or a pharmaceutical composition as described herein for use as a medicament.

Preferably, the immunotoxins described herein are for treatment or prevention of a CMV infection. Immunotoxins are administered in an effective amount to an individual in the need thereof. The individual is any person having a CMV infection or any person in the risk of getting a CMV infection.

Thus, a further aspect of the present invention relates to an immunotoxin as described herein or a pharmaceutical composition as described herein for use in the treatment or prevention of CMV infections or CMV-associated diseases. An embodiment of the present invention relates to the use of the immunotoxin as described herein, wherein treatment or prevention of CMV infections or CMV-associated diseases is (i) in vivo in patients or (ii) ex vivo in cells or organs.

The immunotoxin may also be a constituent in the preparation of a medicament. Therefore, an embodiment of the present invention relates to the use of the immunotoxin as described herein for the manufacture of a medicament for the treatment or prevention of CMV infections or CMV-associated diseases.

CMV infections may occur in a wide variety of locations within the body, with the two overall grouping being tissues and body fluids. Thus, an embodiment of the present invention relates to the immunotoxin or pharmaceutical composition for use as described herein, wherein the CMV infection is present in:
i) a tissue selected from one or more of the group consisting of retina, cornea, heart, liver, kidney, lung, gastro-intestinal tissue, thymus, spleen, skin and muscle, and/or
ii) a body fluid selected from one or more of the group consisting of saliva, blood, urine, semen and breast milk.

CMV is a very common virus that most individuals are infected with during a life span. Thus, the majority of the world population has most likely produced antibodies against the virus and symptoms subsequent to the primary infection are in most cases absent. However, the virus lies dormant in the host, and as soon as the immune system is weakened, the virus may awake from the latent stage. Thus, CMV is a virus that pose a great risk for individuals with weakened immune systems. Thus, an embodiment of the present invention relates to the immunotoxin or pharmaceutical composition for use as described herein, wherein the CMV infection is an infection in an immuno-compromised patient. Another embodiment of the present invention relates to the immunotoxin or pharmaceutical composition for use as described herein, wherein the CMV infection is an infection in an immuno-compromised patient selected from the group consisting of HIV-patients, neonates and immunosuppressive patients, bone marrow transplant patients, solid organ transplant patients, immune therapy patients, cancer patients, intensive care patients, trauma patients, stem cell patients, gene therapy patients, cell therapy patients, geriatric patients and multimorbid patients. A further embodiment of the present invention relates to the immunotoxin or pharmaceutical composition for use as described herein, wherein the CMV infection is an infection in an individual at risk/or planned of becoming immune compromised. An even further embodiment of the present invention relates to the immunotoxin or pharmaceutical composition for use as described herein, wherein the CMV infection is an infection in a patient suffering from a coronary disease and/or a vascular disease. Yet another embodiment of the present invention relates to the immunotoxin or pharmaceutical composition for use as described herein, wherein the CMV infection is a latent CMV infection.

The immunotoxin may be administered by any conventional route. Therefore, an embodiment of the present invention relates to the immunotoxin or pharmaceutical composition for use as described herein, wherein the immunotoxin or pharmaceutical composition is administered via a route selected from one or more of the group consisting of oral, parenteral, intravenous, intradermal, subcutaneous, and topical administration. Another embodiment of the present invention relates to the immunotoxin or pharmaceutical composition for use as described herein, wherein the immunotoxin or pharmaceutical composition is administered to cells or organs ex vivo.

The immunotoxin may be part of a combination treatment, wherein one or more additional therapeutic agents is administered. Therefore, an embodiment of the present invention relates to the immunotoxin or pharmaceutical composition for use as described herein, wherein the immunotoxin or pharmaceutical composition is for simultaneous, separate or sequential administration with one or more additional therapeutic agents. Another embodiment of the present invention relates to the immunotoxin or pharmaceutical composition for use as described herein, wherein the therapeutic agents are selected from the group consisting of anti-viral agents, immunosuppressive agents and modulatory agents.

The immunotoxin may be packed together with other therapeutic agents for easy administration as a combination therapy. Therefore, an aspect of the present invention relates to a kit comprising:
i) an immunotoxin as described herein or a pharmaceutical composition as described herein,
ii) one or more additional therapeutic agents, and
iii) optionally, instructions for use,

wherein i) and ii) are for simultaneous, separate or sequential administration.

The immunotoxins are preferably produced using a recombinant expression system. Suitable recombinant expression systems would be known to the person skilled in the art. For recombinant expression is required; nucleic acids encoding the peptide sequences of interest, expression vectors and an expression system (cell line for recombinant expression). Therefore, an aspect of the present invention relates to a nucleic acid sequence comprising a sequence encoding an immunotoxin as described herein. An embodiment of the present invention relates to a nucleic acid, wherein the nucleic acid sequence selected from:
i) SEQ ID NO:20, or
ii) a nucleic acid sequence having at least 90% sequence identity to SEQ ID NO:20, such as at least 95%, such as at least 96%, such as at least 97%, such as at least 98%, such as at least 99% sequence identity to SEQ ID NO:20.

Nucleic acids encoding individual parts of the immunotoxins are assembled in an expression vector for introduction into an expression system, i.e. a cell. Therefore, another aspect of the present invention relates to a recombinant expression vector comprising a nucleotide sequence as described herein operably linked to one or more control sequences suitable for directing the production of the immunotoxin in a suitable host.

The expression vector is introduced into a host cell using any conventional method and conditions are adjusted to favor recombinant expression of the peptide sequence of interest. Thus, an aspect of the present invention relates to a recombinant host cell comprising an expression vector as described herein. Another aspect of the present invention relates to a method of producing the immunotoxin as described herein comprising the steps of:
i) providing a host cell as described herein,
ii) cultivating said host cell under conditions suitable for the expression of said immunotoxin; and
iii) isolating said immunotoxin.

An embodiment of the present invention relates to a method as described herein, wherein the host cell is either eukaryotic or prokaryotic.

It should be noted that embodiments and features described in the context of one of the aspects of the present invention also apply to the other aspects of the invention. Embodiments and features of the present invention are also outlined in the following items.

Items

1. An immunotoxin comprising:
i) a targeting moiety comprising an amino acid sequence selected from:
   a) SEQ ID NO:1, or
   b) an amino acid sequence having at least 80% sequence identity to SEQ ID NO:1, and
ii) a toxin,

wherein the amino acid residue in position 49 is replaced by an alanine (A) residue and the amino acid residues in positions 1-6 are replaced with the amino acid sequence ILDNGVS in the N-terminal end.

2. The immunotoxin according to item 1, wherein the amino acid sequence of (b) has at least 90% sequence identity to SEQ ID NO:1, such as at least 95%, such as at least 96%, such as at least 97%, such as at least 98%, such as at least 99% sequence identity to SEQ ID NO:1.

3. The immunotoxin according to any one of the preceding items, wherein the targeting moiety comprises SEQ ID NO:2.

4. The immunotoxin according to any one of the preceding items, wherein the amino acid of said targeting moiety further comprises a methionine (M) residue as the first amino acid from the N-terminal end.

5. The immunotoxin according to any one of the preceding items, wherein the targeting moiety binds to the US28 receptor.

6. The immunotoxin according to item 5, wherein the US28 receptor comprises an amino acid sequence selected from:
i) SEQ ID NO:3, or
ii) an amino acid sequence having at least 80% sequence identity to SEQ ID NO:3.

7. The immunotoxin according to any one of the preceding items, wherein the immunotoxin binds US28 with a Kᵢ of 10⁻⁷ M or less, such as 10⁻⁸ M or less, such as 10⁻⁹ M or less, such as 10⁻¹⁹ M or less, such as 10⁻¹¹ M or less.

8. The immunotoxin according to any one of the preceding items, wherein the affinity of the immunotoxin for the human homologous receptor CX3CR1 is reduced as compared to the affinity of CX3CL1 (SEQ ID NO:1) for CX3CR1, such as at least 100-fold, such as at least 150-fold, such as 200-fold, or such as at least 250-fold reduced.

9. The immunotoxin according to any one of the preceding items, wherein the immunotoxin binds the CX3CR1 with a Kᵢ of 10⁻⁶ or more.

10. The immunotoxin according to any one of the preceding items, wherein the immunotoxin has increased affinity for US28 as compared to the affinity for CX3CR1, such as at least 75-fold, such as at least 100-fold, such as at least 150-fold, such as 200-fold, or such as at least 250-fold increased affinity.

11. The immunotoxCX3CR1, such as at least 75-fold, such as at least 100-fold, such as at least 150-fold, such as 200-fold, or such as at least 250-fold increased affinity.

11. The immunotoxin according to any one of items 8-10, wherein the CX3CR1 receptor comprises an amino acid sequence according to SEQ ID NO:4.

12. The immunotoxin according to any one of the preceding items, wherein the immunotoxin is internalized subsequent to binding US28.

13. The immunotoxin according to any one of the preceding items, wherein the toxin is selected from one or more of the group consisting of Pseudomonas exotoxin A, gelonin, bouganin, saporin, ricin, ricin A chain, bryodin, diphtheria, restrictocin, diphtheria toxin, and fragments or variants thereof.

14. The immunotoxin according to any one of the preceding items, wherein the toxin is Pseudomonas exotoxin A (SEQ ID NO:5) or a fragment thereof.

15. The immunotoxin according to item 14, wherein the toxin comprises one or more fragments selected from the group consisting of the binding domain (domain II, SEQ ID NO:6), intermediate domain (domain Ib, SEQ ID NO:7) and the ADP-ribosylating domain (domain III, SEQ ID NO:8) of Pseudomonas exotoxin A.

16. The immunotoxin according to item 15, wherein the C-terminal amino acid sequence REDLK (SEQ ID NO:15) of the ADP-ribosylating domain is replaced by the amino acid sequence KDEL (SEQ ID NO:16).

17. The immunotoxin according to item 16, wherein the toxin is PE38KDEL (SEQ ID NO:9).

18. The immunotoxin according to any one of the preceding items, wherein the immunotoxin comprises SEQ ID NO:9 and SEQ ID NO:2.

19. The immunotoxin according to any one of the preceding items, wherein the immunotoxin comprises SEQ ID NO:10.

20. The immunotoxin according to any one of the preceding items, wherein the immunotoxin has increased potency against cell expressing US28 as compared to the potency against cells expressing CX3CR1, such as at least 150-fold, such as 175-fold, or such as at least 200-fold, such as at least 225-fold, such as at least 250-fold increased potency.

21. A pharmaceutical composition comprising an immunotoxin according to any one of the preceding items or a pharmaceutically acceptable salt thereof.

22. The pharmaceutical composition according to item 21, wherein the composition comprises a pharmaceutically acceptable carrier, diluent and/or excipient.

23. An immunotoxin according to any one of items 1-20 or a pharmaceutical composition according to any one of items 21 or 22 for use as a medicament.

24. An immunotoxin according to any one of items 1-20 or a pharmaceutical composition according to any one of items 21 or 22 for use in the treatment or prevention of CMV infections or CMV-associated diseases.

25. The immunotoxin or pharmaceutical composition for use according to item 24, wherein the CMV infection is present in:
i) a tissue selected from one or more of the group consisting of retina, cornea, heart, liver, kidney, lung, gastro-intestinal tissue, thymus, spleen, skin and muscle, and/or
ii) a body fluid selected from one or more of the group consisting of saliva, blood, urine, semen and breast milk.

26. The immunotoxin or pharmaceutical composition for use according to any one of items 24 or 25, wherein the CMV infection is an infection in an immuno-compromised patient selected from the group consisting of HIV-patients, neonates and immunosuppressive patients, bone marrow transplant patients, solid organ transplant patients, immune therapy patients, cancer patients, intensive care patients, trauma patients, stem cell patients, gene therapy patients, cell therapy patients, geriatric patients and multimorbid patients.

27. The immunotoxin or pharmaceutical composition for use according to any one of items 24 or 25, wherein the CMV infection is an infection in a patient suffering from a coronary disease and/or a vascular disease.

28. The immunotoxin or pharmaceutical composition for use according to any one of items 24-27, wherein the CMV infection is a latent CMV infection.

29. The immunotoxin or pharmaceutical composition for use according to any one of items 23-28, wherein the immunotoxin or pharmaceutical composition is administered via a route selected from one or more of the group consisting of oral, parenteral, intravenous, intradermal, subcutaneous, and topical administration.

30. The immunotoxin or pharmaceutical composition for use according to any one of items 23-29, wherein the immunotoxin or pharmaceutical composition is for simultaneous, separate or sequential administration with one or more additional therapeutic agents.

31. The immunotoxin or pharmaceutical composition for use according to item 30, wherein the therapeutic agents are selected from the group consisting of anti-viral agents, immunosuppressive agents and modulatory agents.

32. A kit comprising:
i) an immunotoxin according to any one of items 1-20 or a pharmaceutical composition according to any one of items 21 or 22,
ii) one or more additional therapeutic agents, and
iii) optionally, instructions for use,

wherein i) and ii) are for simultaneous, separate or sequential administration.

33. A nucleic acid sequence comprising a sequence encoding an immunotoxin according to any one of items 1-20.

34. A recombinant expression vector comprising a nucleotide sequence according to item 33 operably linked to one or more control sequences suitable for directing the production of the immunotoxin in a suitable host.

35. A recombinant host cell comprising an expression vector according to item 34.

36. A method of producing the immunotoxin according to any one of items 1-20 comprising the steps of:
i) providing a host cell according to item 35,
ii) cultivating said host cell under conditions suitable for the expression of said immunotoxin; and
iii) isolating said immunotoxin.

All patent and non-patent references cited in the present application, are hereby incorporated by reference in their entirety.

The invention will now be described in further details in the following non-limiting examples.

### EXAMPLES

### Example 1

### Preparation of Fusion Proteins SYN000, SYN001, SYN003 and SYN004

Structure of Immunotoxins

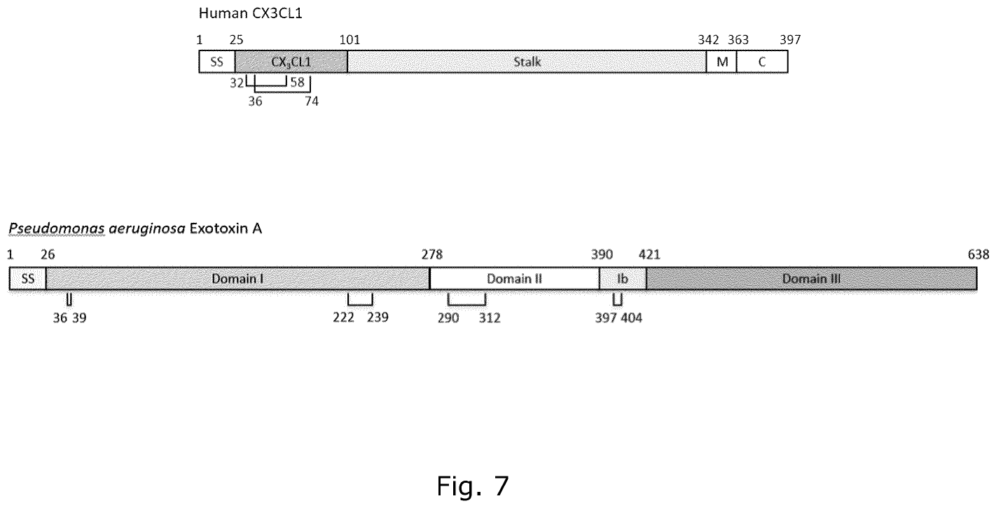

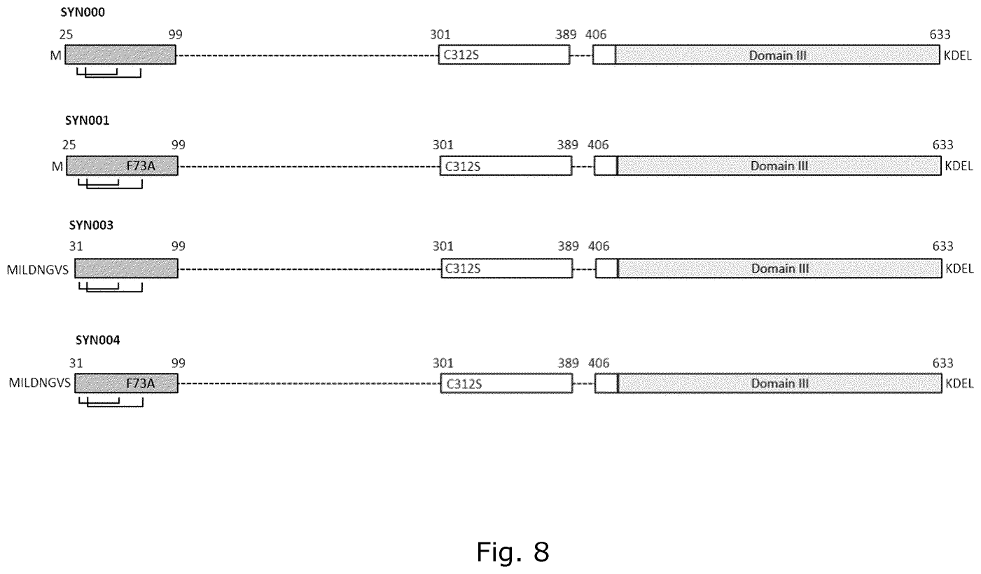

Herein are disclosed a series of immunotoxins designed to specifically target US28. They are based on the chemokine CX3CL1 (fractalkine) and Exotoxin A of Pseudomonas aeruginosa (both depicted in FIG. 7). Drug substance candidates SYN000 (SEQ ID NO:17), SYN001 (SEQ ID NO:18), SYN003 (SEQ ID NO:19) and SYN004 (SEQ ID NO:10) are single polypeptide chains consisting of ca. 400 amino acids with domain structures as depicted in FIG. 8.

Production of Immunotoxins

The immunotoxins are produced as insoluble protein aggregates (inclusion bodies, IB's) in Escherichia coli (E. coli). The drug substance manufacturing process consists of three phases of processing: cell culture and harvest, recovery and purification. The E. coli culture step is where IB's are produced containing high levels of the drug substance. The IB's are recovered by a series of washes and centrifugations. The purification process is comprised of IB solubilization, refolding by dialysis against a phosphate buffer containing a redox-couple followed by AEX- and GF-chromatographic methods to obtain a pure drug substance.

### Example 2

### Affinity (Kᵢ values) of SYN000, SYN001, SYN003 and SYN004 to US28 and CX3CR1

Receptor Competition Binding

Stable inducible clones of US28-HEK293 cells and CX3CR1-HEK293 cells were grown in a humidified incubator at 10% CO₂ and 37° C. in Dulbecco's modified Eagle's medium (DMEM) GlutaMAX (GIBCOR) with 10% fetal bovine serum and 180 units/mL penicillin and 45 μg/mL streptomycin. The cells were seeded at 10,000 cells per well in poly-D-lysine (Invitrogen)-coated 96-well tissue culture plates (Nunc) in 100 μL growth medium. One day after seeding, US28 and CX3CR1 expression was induced by tetracycline (5 ng/mL) to obtain 5-10% specific binding. Competition binding studies were performed in duplicates 1 d after induction. In brief, the cells were washed twice in binding buffer consisting of 4-(2-hydroxyethyl)-1-piperazineethanesulfonic acid (HEPES) buffer (50 mM HEPES, 5 mM MgCl2, 1 mM CaCl2, pH 7.2) and 0.5% bovine serum albumin. The cells were thereafter incubated for 3 h at 4° C. with ˜25 pM of ¹²⁵I-CX3CL1 (or ¹²⁵1-CCL2) plus 5 μL of the unlabeled ligand, i.e. the immunotoxin or the homologous chemokine, in 100 μL binding buffer. Afterward cells were washed twice with 4° C. binding buffer supplemented with 0.5 M NaCl. As the last step cells were lysed with 180 μL of 200 mM NaOH and 1% sodium dodecylsulfate lysis buffer. Data were collected using a Gamma-counter (1470 Wizard). The Kᵢ values were calculated from the IC50 (the concentration of the unlabeled ligands that reveals 50% displacement of the specifically bound radioligand), the [L] (the radioligand concentration), the K_{d} (the dissociation constant for the radioligand) using the Cheng-Prusoff equation, formula (I):

$${K}_{i} = \frac{IC 50}{1 + ( \frac{[ L ]}{{K}_{d}} )} \qquad Formula ( I )$$

Results

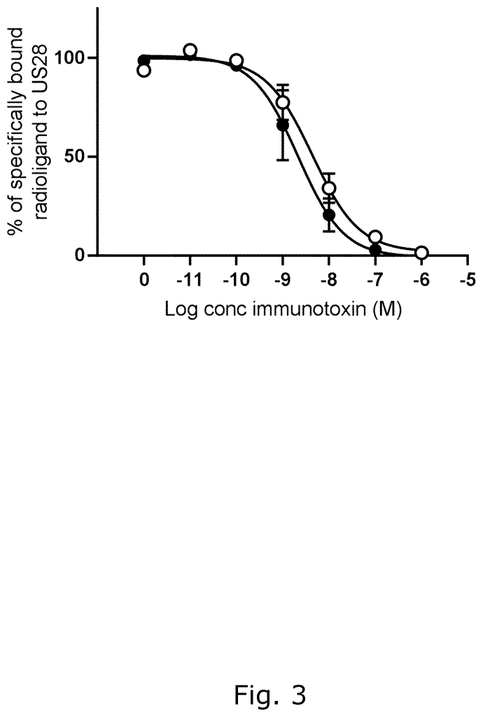

¹²⁵I-CCL2 as radioligand: SYN000 binds with an affinity (Log Kᵢ) of −8.67, SYN001 of -7.96, SYN003 of −8.36 and SYN004 of −7.36 to US28 respectively, as shown in FIGS. 1 and 3, and table 1.

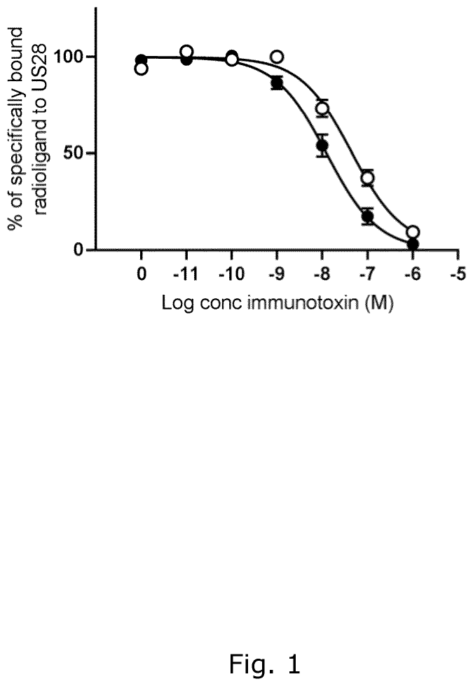

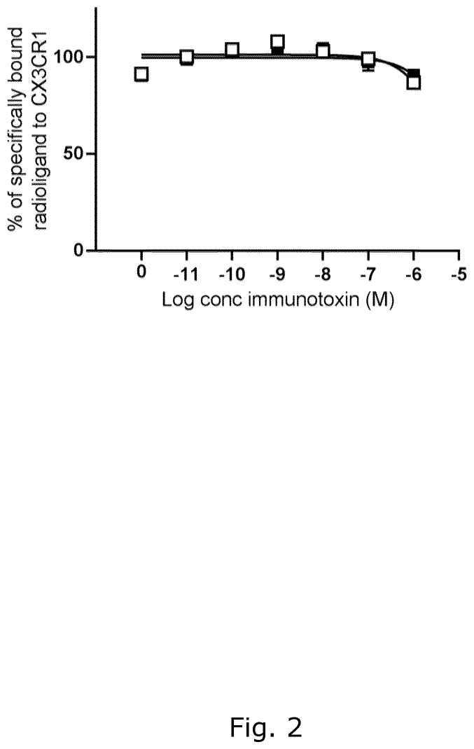

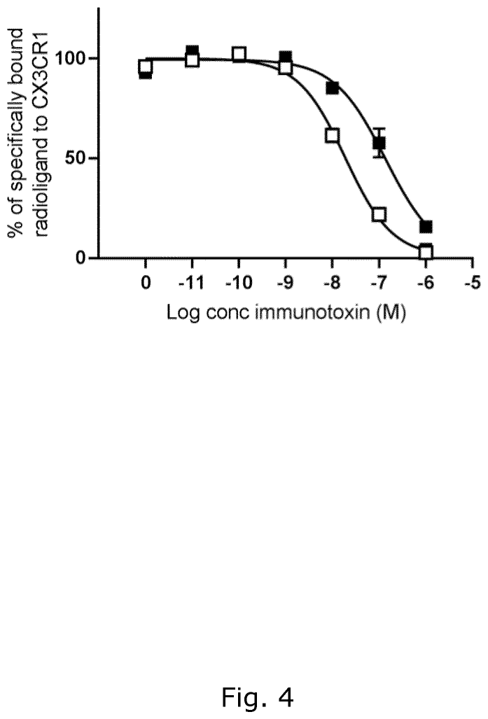

¹²⁵I-CX3CL1 as radioligand: SYN000 binds with an affinity (Log Kᵢ) of −6.86, SYN001 of −5.00, SYN003 of −7.72 and SYN004 of −5.22 to CX3CR1 respectively, as shown in FIGS. 2 and 4, and table 1.

TABLE 1
Receptor competition binding

| | SYN000 | SYN000 | SYN003 | SYN003 | SYN001 | SYN001 | SYN004 | SYN004 |
|---|---|---|---|---|---|---|---|---|
| | US28 | CX3CR1 | US28 | CX3CR1 | US28 | CX3CR1 | US28 | CX3CR1 |
| Log Kᵢ | −8.67 ± | −6.86 ± | −8.36 ± | −7.72 ± | −7.96 ± | −5.00 ± | −7.36 ± | −5.22 ± |
| | 0.16 | 0.07 | 0.12 | 0.08 | 0.09 | 0.19 | 0.13 | 0.13 |
| Ratio | 64.6 | | 4.37 | | 912 | | 138 | |
| US28/ | | | | | | | | |
| CX3CR1 | | | | | | | | |
| Ratio | 0.07 | | | | 0.15 | | | |
| F1-alone | | | | | | | | |
| F1-F49A | | | | | | | | |

The introduced N-terminal amino acid sequence seems to modestly lower the affinity to US28 2-fold in; SYN003 (log Kᵢ −8.36) compared to SYN000 (log Kᵢ −8.67), and 4-fold; SYN004 (log Kᵢ −7.36) compared to SYN001 (log Kᵢ −7.96). In contrast the N-terminal amino acid sequence modestly increase the affinity to CX3CR1 7-fold; SYN003 (log Kᵢ −7.72) compared to SYN000 (log Kᵢ −6.86), and 1,7-fold; SYN004 (log Kᵢ −5.22) compared to SYN001 (log Kᵢ −5.00).

Conclusion

Overall, the introduction of the N-terminal amino acid sequence (F1 mutation) in the immunotoxins decreases affinity to US28 and increases affinity to CX3CR1. Consequently, SYN003 and SYN004 are less selective in its preference for US28 over CX3CR1 than SYN000 and SYN001, respectively (fold changes are less than 1, see table 1).

### Example 3

### Potency of SYN000, SYN001, SYN003 and SYN004 Against Induced US28 HEK293 Cells and Induced CX3CR1 HEK293 Cells

In Vitro Potency

Stable inducible clones of US28-HEK293 and CX3CR1-HEK293 cells and naïve HEK293 cells were grown in a humidified incubator at 10% CO₂ and 37° C. in Dulbecco's modified Eagle's medium (DMEM) GlutaMAX (GIBCOR) with 10% fetal bovine serum and 180 units/mL penicillin and 45 μg/mL streptomycin. The cells were seeded at 11,000 cells per well in poly-D-lysine (Invitrogen)-coated 96-well tissue culture plates (Nunc) in 100 μL growth medium. Receptor expression was induced 24 h after seeding using 0.125 μg/mL (US28) and 0.5 μg/mL (CX3CR1) tetracycline. The different concentrations of the indicated immunotoxin (0.01 pM- 0.1 pM) and buffer (mock treatment) were added 1 d after receptor induction in a final volume of 100 μL growth medium and were incubated for 24 h at 37° C. To estimate cell viability, the cells were incubated with AlamarBlue (Invitrogen) mixed 1:10 with growth medium, 100 μL per well, for 4 h at 37° C. The data was collected using the FlexStation 3 (Molecular Devices) plate reader where the fluorescence was measured by excitation at 540 nm wavelength and reading the emission at 585nm wavelength. Cell viability was determined at different concentrations of immunotoxin and the IC50 value was extracted at 50% cell viability. Reference values corresponding were obtained in the presence of Cycloheximide (0% cell viability) and in the absence of immunotoxin or Cycloheximide (100% cell viability).

Results

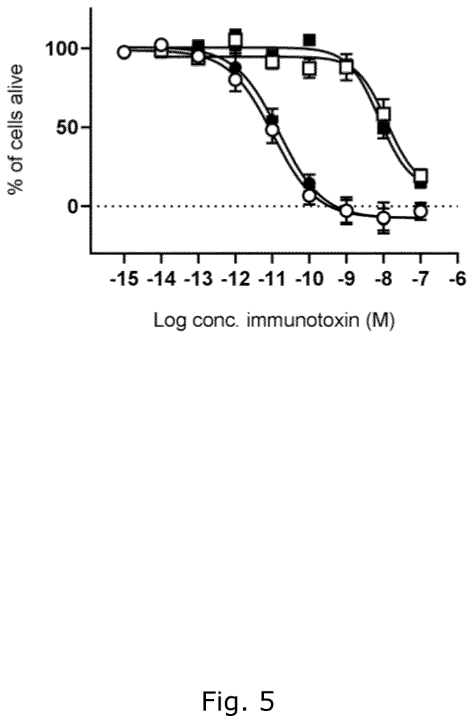

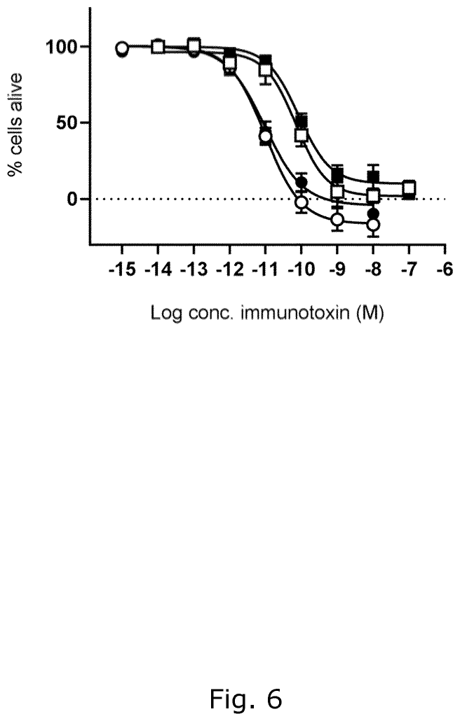

FIGS. 5 and 6, and table 2 show the potency of SYN000, SYN001, SYN003 and SYN004. On US28, the potencies are similar with a tendency of SYN000, SYN003 and SYN004 having slightly higher potency on US28 than SYN001. On CX3CR1, the potencies vary greatly, with SYN003 and SYN000 having higher potencies than SYN001 and SYN004, and SYN004 having slightly lower potency on CX3CR1 than SYN001.

The potencies may be used to calculate the killing specificity of each immunotoxin, i.e. the ability of the immunotoxin to specifically kill cells expressing US28 over cells expressing CX3CR1. The killing specificities are reported as fold change in table and show an 8.91-fold change for SYN000, a 7.08-fold change for SYN003, a 501-fold change for SYN001 and a 1175-fold change for SYN004.

TABLE 2
Potency of immunotoxins

| | SYN000 | SYN000 | SYN003 | SYN003 | SYN001 | SYN001 | SYN004 | SYN004 |
|---|---|---|---|---|---|---|---|---|
| | US28 | CX3CR1 | US28 | CX3CR1 | US28 | CX3CR1 | US28 | CX3CR1 |
| Log | −11.03 ± | −10.08 ± | −11.01 ± | −10.16 ± | −10.82 ± | −8.12 ± | −10.99 ± | −7.92 ± |
| IC50 | 0.15 | 0.10 | 0.11 | 0.12 | 0.15 | 0.15 | 0.17 | 0.18 |
| Ratio | 8.91 | | 7.08 | | 501 | | 1175 | |
| US28/ | | | | | | | | |
| CX3CR1 | | | | | | | | |
| Ratio | 0.82 | | | | | 2.35 | | |
| F1-alone | | | | | | | | |
| F1-F49A | | | | | | | | |

Conclusion

It is demonstrated that the addition of the F1 sequence to SYN000 (creating SYN003) is not contributing to increased killing specificity (ratio is less than 1, see table 2), whereas the addition of the Fl sequence to SYN001 (resulting in SYN004) increase killing specificity 2.35-fold. Consequently, despite having decreased selectivity (see example 2), SYN004 surprisingly represents a significantly improved immunotoxin, i.e. with increased killing specificity.

### REFERENCES

WO 2008/003327

1. An immunotoxin comprising:
i) a targeting moiety comprising an amino acid sequence selected from:
a) SEQ ID NO:1, or
b) an amino acid sequence having at least 80% sequence identity to SEQ ID NO:1, and
ii) a toxin,
wherein the amino acid residue in position 49 is replaced by an alanine (A) residue and the amino acid residues in positions 1-6 are replaced with the amino acid sequence ILDNGVS in the N-terminal end.

2. The immunotoxin according to claim 1, wherein the amino acid sequence of (b) has at least 90% sequence identity to SEQ ID NO:1, optionally at least 95%, optionally at least 96%, optionally at least 97%, optionally at least 98%, or optionally at least 99% sequence identity to SEQ ID NO:1.

3. The immunotoxin according to claim 1, wherein the targeting moiety binds to the US28 receptor.

4. The immunotoxin according to claim 3, wherein the US28 receptor comprises an amino acid sequence selected from:
i) SEQ ID NO:3, or
ii) an amino acid sequence having at least 80% sequence identity to SEQ ID NO:3.

5. The immunotoxin according to claim 1, wherein the affinity of the immunotoxin for the human homologous receptor CX3CR1 is reduced as compared to the affinity of CX3CL1 (SEQ ID NO:1) for CX3CR1, optionally at least 100-fold, optionally at least 150-fold, optionally 200-fold, or optionally as at least 250-fold reduced affinity.

6. The immunotoxin according to claim 1, wherein the immunotoxin has increased affinity for US28 as compared to the affinity for CX3CR1, optionally at least 75-fold, optionally at least 100-fold, optionally at least 150-fold, optionally 200-fold, or optionally at least 250-fold increased affinity.

7. The immunotoxin according to claim 5, wherein the CX3CR1 receptor comprises an amino acid sequence according to SEQ ID NO:4.

8. The immunotoxin according to claim 1, wherein the toxin is selected from one or more of the group consisting of Pseudomonas exotoxin A, gelonin, bouganin, saporin, ricin, ricin A chain, bryodin, diphtheria, restrictocin, diphtheria toxin, and fragments or variants thereof.

9. The immunotoxin according to claim 1, wherein the toxin is Pseudomonas exotoxin A (SEQ ID NO:5) or a fragment thereof.

10. The immunotoxin according to claim 1, wherein the immunotoxin comprises SEQ ID NO:10.

11. The immunotoxin according to claim 1, wherein the immunotoxin has increased potency against cell expressing US28 as compared to the potency against cells expressing CX3CR1, optionally at least 150-fold, optionally 175-fold, optionally at least 200-fold, optionally at least 225-fold, optionally at least 250-fold increased potency.

12. A pharmaceutical composition comprising an immunotoxin according to claim 1 or a pharmaceutically acceptable salt thereof.

13. An immunotoxin according to claim 1 for use as a medicament.

14. An immunotoxin according to claim 1 for use in the treatment or prevention of CMV infections or CMV-associated diseases.

15. A kit comprising:
i) an immunotoxin according to claim 1,
ii) one or more additional therapeutic agents, and
iii) optionally, instructions for use,
wherein i) and ii) are for simultaneous, separate or sequential administration.

16. A pharmaceutical composition according to claim 12 for use as a medicament.

17. A pharmaceutical composition according to claim 12 for use in the treatment or prevention of CMV infections or CMV-associated disease.

18. A kit, comprising:
i) a pharmaceutical composition according to claim 12,
ii) one or more additional therapeutic agents, and
iii) optionally, instructions for use,
wherein i) and ii) are for simultaneous, separate or sequential administration.


SEQUENCE LISTING (20 sequences)

SEQ ID NO: 1 - protein, 75 aa, Homo sapiens
  CX3CL1 (1)..(1)
QHHGVTKCNITCSKMTSKIPVALLIHYQQNQASCGKRAIILETRQHRLFCADPKEQWVKDAMQHLDRQAAALTRN

SEQ ID NO: 2 - protein, 76 aa, Homo sapiens
  CX3CL1 with F1 + F49A (1)..(1)
ILDNGVSKCNITCSKMTSKIPVALLIHYQQNQASCGKRAIILETRQHRLACADPKEQWVKDAMQHLDRQAAALTRN

SEQ ID NO: 3 - protein, 354 aa, Cytomegalovirus
  US28 (1)..(1)
MTPTTTTAELTTEFDYDEAATPCVFTDVLNQSKPVTLFLYGVVFLFGSIGNFLVIFTITWRRRIQCSGDVYFINLAAADLLFVCTLPLWMQYLLDHNSLASVPCTLLTACFYVAMFASLCFITEIALDRYYAIVYMRYRPVKQACLFSIFWWIFAVIIAIPHFMVVTKKDNQCMTDYDYLEVSYPIILNVELMLGAFVIPLSVISYCYYRISRIVAVSQSRHKGRIVRVLIAVVLVFIIFWLPYHLTLFVDTLKLLKWISSSCEFERSLKRALILTESLAFCHCCLNPLLYVFVGTKFRQELHCLLAEFRQRLFSRDVSWYHSMSFSRRSSPSRRETSSDTLSDEVCRVSQIIP

SEQ ID NO: 4 - protein, 355 aa, Homo sapiens
  CX3CR1 (1)..(1)
MDQFPESVTENFEYDDLAEACYIGDIVVFGTVFLSIFYSVIFAIGLVGNLLVVFALTNSKKPKSVTDIYLLNLALSDLLFVATLPFWTHYLINEKGLHNAMCKFTTAFFFIGFFGSIFFITVISIDRYLAIVLAANSMNNRTVQHGVTISLGVWAAAILVAAPQFMFTKQKENECLGDYPEVLQEIWPVLRNVETNFLGFLLPLLIMSYCYFRIIQTLFSCKNHKKAKAIKLILLVVIVFFLFWTPYNVMIFLETLKLYDFFPSCDMRKDLRLALSVTETVAFSHCCLNPLIYAFAGEKFRRYLYHLYGKCLAVLCGRSVHVDFSSSESQRSRHGSVLSSNFTYHTSDGDALLLL

SEQ ID NO: 5 - protein, 638 aa, Pseudomonas aeruginosa
  Full-length Pseudomonas exotoxin A (1)..(1)
MHLTPHWIPLVASLGLLAGGSFASAAEEAFDLWNECAKACVLDLKDGVRSSRMSVDPAIADTNGQGVLHYSMVLEGGNDALKLAIDNALSITSDGLTIRLEGGVEPNKPVRYSYTRQARGSWSLNWLVPIGHEKPSNIKVFIHELNAGNQLSHMSPIYTIEMGDELLAKLARDATFFVRAHESNEMQPTLAISHAGVSVVMAQAQPRREKRWSEWASGKVLCLLDPLDGVYNYLAQQRCNLDDTWEGKIYRVLAGNPAKHDLDIKPTVISHRLHFPEGGSLAALTAHQACHLPLETFTRHRQPRGWEQLEQCGYPVQRLVALYLAARLSWNQVDQVIRNALASPGSGGDLGEAIREQPEQARLALTLAAAESERFVRQGTGNDEAGAASADVVSLTCPVAAGECAGPADSGDALLERNYPTGAEFLGDGGDISFSTRGTQNWTVERLLQAHRQLEERGYVFVGYHGTFLEAAQSIVFGGVRARSQDLDAIWRGFYIAGDPALAYGYAQDQEPDARGRIRNGALLRVYVPRSSLPGFYRTGLTLAAPEAAGEVERLIGHPLPLRLDAITGPEEEGGRLETILGWPLAERTVVIPSAIPTDPRNVGGDLDPSSIPDKEQAISALPDYASQPGKPPREDLK

SEQ ID NO: 6 - protein, 112 aa, Pseudomonas aeruginosa
  Pseudomonas exotoxin A domain II (1)..(1)
GGSLAALTAHQACHLPLETFTRHRQPRGWEQLEQCGYPVQRLVALYLAARLSWNQVDQVIRNALASPGSGGDLGEAIREQPEQARLALTLAAAESERFVRQGTGNDEAGAAS

SEQ ID NO: 7 - protein, 40 aa, Pseudomonas aeruginosa
  Pseudomonas exotoxin A domain Ib (1)..(1)
ADVVSLTCPVAAGECAGPADSGDALLERNYPTGAEFLGDG

SEQ ID NO: 8 - protein, 209 aa, Pseudomonas aeruginosa
  Pseudomonas exotoxin A domain III (1)..(1)
GDISFSTRGTQNWTVERLLQAHRQLEERGYVFVGYHGTFLEAAQSIVFGGVRARSQDLDAIWRGFYIAGDPALAYGYAQDQEPDARGRIRNGALLRVYVPRSSLPGFYRTGLTLAAPEAAGEVERLIGHPLPLRLDAITGPEEEGGRLETILGWPLAERTVVIPSAIPTDPRNVGGDLDPSSIPDKEQAISALPDYASQPGKPPREDLK

SEQ ID NO: 9 - protein, 344 aa, Pseudomonas aeruginosa
  PE38KDEL (1)..(1)
GGSLAALTAHQACHLPLETFTRHRQPRGWEQLEQCGYPVQRLVALYLAARLSWNQVDQVIRNALASPGSGGDLGEAIREQPEQARLALTLAAAESERFVRQGTGNDEAGAASGPADSGDALLERNYPTGAEFLGDGGDISFSTRGTQNWTVERLLQAHRQLEERGYVFVGYHGTFLEAAQSIVFGGVRARSQDLDAIWRGFYIAGDPALAYGYAQDQEPDARGRIRNGALLRVYVPRSSLPGFYRTGLTLAAPEAAGEVERLIGHPLPLRLDAITGPEEEGGRLETILGWPLAERTVVIPSAIPTDPRNVGGDLDPSSIPDKEQAISALPDYASQPGKPPKDEL

SEQ ID NO: 10 - protein, 398 aa, Artificial sequence
  Immunotoxin SYN004
  SYN004 (1)..(1)
MILDNGVSKCNITCSKMTSKIPVALLIHYQQNQASCGKRAIILETRQHRLACADPKEQWVKDAMQHLDRQAAALTRNRQPRGWEQLEQSGYPVQRLVALYLAARLSWNQVDQVIRNALASPGSGGDLGEAIREQPEQARLALTLAAAESERFVRQGTGNDEAGAASGPADSGDALLERNYPTGAEFLGDGGDISFSTRGTQNWTVERLLQAHRQLEERGYVFVGYHGTFLEAAQSIVFGGVRARSQDLDAIWRGFYIAGDPALAYGYAQDQEPDARGRIRNGALLRVYVPRSSLPGFYRTGLTLAAPEAAGEVERLIGHPLPLRLDAITGPEEEGGRLETILGWPLAERTVVIPSAIPTDPRNVGGDLDPSSIPDKEQAISALPDYASQPGKPPKDEL

SEQ ID NO: 11 - protein, 77 aa, Homo sapiens
  CX3CL1 with F1 + F49A and N-terminal M (1)..(1)
MILDNGVSKCNITCSKMTSKIPVALLIHYQQNQASCGKRAIILETRQHRLACADPKEQWVKDAMQHLDRQAAALTRN

SEQ ID NO: 12 - DNA, 1065 bp, Cytomegalovirus
  US28 gene sequence (1)..(1)
atgacgccgacgacgacgaccgcggaactcacgacggagtttgactacgatgaagccgcgactccttgtgttttcaccgacgtgcttaatcaatcaaagccggttacgttgtttctgtacggcgttgtctttctgttcggttccatcggcaacttcttggtgatcttcaccatcacctggcgacgtcggattcaatgctccggcgatgtttactttatcaacctcgcggccgccgatttgcttttcgtttgtacactacctctgtggatgcaatacctcctagatcacaactccctagccagcgtgccgtgtacgttactcactgcctgtttctacgtggctatgtttgccagtttgtgttttatcacggagattgcactcgatcgctactacgctattgtttacatgagatatcggcctgtaaaacaggcctgccttttcagtattttttggtggatctttgccgtgatcatcgccattccacactttatggtggtgaccaaaaaaaacaatcaatgtatgaccgactacgactacttagaggtcagttacccgatcatcctcaacgtagaactcatgctcggtgctttcgtgatcccgctcagtgttatcagctactgctactaccgcatttccagaatcgttgcggtgtctcagtcgcgccacaaaggtcgcattgtacgggtacttatagcggtcgtgcttgtctttatcatcttttggctgccgtaccacctaacgctgtttgtggacacgttaaaactcctcaaatggatctccagcagctgcgagttcgaaagatcgctcaaacgtgcgctcatcttgaccgagtcgctcgccttttgtcactgttgtctcaatccgctgctgtacgtcttcgtgggcaccaagtttcggcaagaactgcactgtctgctggccgagtttcgccagcgactcttttcccgcgatgtatcctggtaccacagcatgagcttttcgcgtcggagctcgccgagccgaagagagacatcttccgacacgctgtccgacgaggtgtgtcgcgtctcacaaattataccgtaa

SEQ ID NO: 13 - protein, 7 aa, Artificial sequence
  N-terminal end of targeting moiety with F1
ILDNGVS

SEQ ID NO: 14 - protein, 6 aa, Artificial sequence
  N-terminal end of targeting moiety without F1
QHHGVT

SEQ ID NO: 15 - protein, 5 aa, Artificial sequence
  C-terminal end of the ADP-ribosylating domain of PE
REDLK

SEQ ID NO: 16 - protein, 4 aa, Artificial sequence
  Modified C-terminal end of the ADP-ribosylating domain of PE
KDEL

SEQ ID NO: 17 - protein, 397 aa, Artificial sequence
  Immunotoxin SYN000
  SYN000 (1)..(1)
MQHHGVTKCNITCSKMTSKIPVALLIHYQQNQASCGKRAIILETRQHRLFCADPKEQWVKDAMQHLDRQAAALTRNRQPRGWEQLEQSGYPVQRLVALYLAARLSWNQVDQVIRNALASPGSGGDLGEAIREQPEQARLALTLAAAESERFVRQGTGNDEAGAASGPADSGDALLERNYPTGAEFLGDGGDISFSTRGTQNWTVERLLQAHRQLEERGYVFVGYHGTFLEAAQSIVFGGVRARSQDLDAIWRGFYIAGDPALAYGYAQDQEPDARGRIRNGALLRVYVPRSSLPGFYRTGLTLAAPEAAGEVERLIGHPLPLRLDAITGPEEEGGRLETILGWPLAERTVVIPSAIPTDPRNVGGDLDPSSIPDKEQAISALPDYASQPGKPPKDEL

SEQ ID NO: 18 - protein, 397 aa, Artificial sequence
  Immunotoxin SYN001
  SYN001 (1)..(1)
MQHHGVTKCNITCSKMTSKIPVALLIHYQQNQASCGKRAIILETRQHRLACADPKEQWVKDAMQHLDRQAAALTRNRQPRGWEQLEQSGYPVQRLVALYLAARLSWNQVDQVIRNALASPGSGGDLGEAIREQPEQARLALTLAAAESERFVRQGTGNDEAGAASGPADSGDALLERNYPTGAEFLGDGGDISFSTRGTQNWTVERLLQAHRQLEERGYVFVGYHGTFLEAAQSIVFGGVRARSQDLDAIWRGFYIAGDPALAYGYAQDQEPDARGRIRNGALLRVYVPRSSLPGFYRTGLTLAAPEAAGEVERLIGHPLPLRLDAITGPEEEGGRLETILGWPLAERTVVIPSAIPTDPRNVGGDLDPSSIPDKEQAISALPDYASQPGKPPKDEL

SEQ ID NO: 19 - protein, 398 aa, Artificial sequence
  Immunotoxin SYN003
  SYN003 (1)..(1)
MILDNGVSKCNITCSKMTSKIPVALLIHYQQNQASCGKRAIILETRQHRLFCADPKEQWVKDAMQHLDRQAAALTRNRQPRGWEQLEQSGYPVQRLVALYLAARLSWNQVDQVIRNALASPGSGGDLGEAIREQPEQARLALTLAAAESERFVRQGTGNDEAGAASGPADSGDALLERNYPTGAEFLGDGGDISFSTRGTQNWTVERLLQAHRQLEERGYVFVGYHGTFLEAAQSIVFGGVRARSQDLDAIWRGFYIAGDPALAYGYAQDQEPDARGRIRNGALLRVYVPRSSLPGFYRTGLTLAAPEAAGEVERLIGHPLPLRLDAITGPEEEGGRLETILGWPLAERTVVIPSAIPTDPRNVGGDLDPSSIPDKEQAISALPDYASQPGKPPKDEL

SEQ ID NO: 20 - DNA, 1194 bp, Artificial sequence
  nucleic acid sequence of SYN004
atgatcctggacaacggtgtgagcaagtgcaacattacctgcagcaagatgaccagcaaaatcccggttgcgctgctgattcactaccagcaaaaccaggcgagctgcggcaaacgtgcgatcattctggaaacccgtcagcaccgtctggcgtgcgcggatccgaaggagcaatgggtgaaagacgcgatgcagcatctggatcgtcaagcggcggcgctgacccgtaaccgtcagccgcgtggttgggaacagctggagcaaagcggctacccggtgcaacgtctggttgcgctgtatctggcggcgcgtctgagctggaaccaggtggaccaagttatccgtaacgcgctggcgagcccgggtagcggtggcgatctgggtgaagcgattcgtgaacagccggagcaagcgcgtctggcgctgaccctggcggcggcggaaagcgagcgttttgttcgtcagggtaccggtaacgatgaggcgggtgcggcgagcggtccggcggacagcggtgatgcgctgctggaacgtaactatccgaccggtgcggagttcctgggtgacggtggcgatatcagctttagcacccgtggtacccagaactggaccgttgaacgtctgctgcaggcgcaccgtcaactggaggaacgtggctacgtgttcgttggttatcacggcacctttctggaggcggcgcagagcattgtgttcggtggcgttcgtgcgcgtagccaagacctggatgcgatctggcgtggtttttatattgcgggcgatccggcgctggcgtacggttatgcgcaggaccaagaaccggatgcgcgtggtcgtatccgtaacggtgcgctgctgcgtgtgtatgttccgcgtagcagcctgccgggtttttatcgtaccggcctgaccctggcggcgccggaggcggcgggtgaagtggaacgtctgattggtcacccgctgccgctgcgtctggacgcgattaccggtccggaggaagagggtggccgtctggaaaccattctgggttggccgctggcggagcgtaccgtggttatcccgagcgcgattccgaccgatccgcgtaacgttggtggcgacctggatccgagcagcatcccggacaaggaacaggcgattagcgcgctgccggattatgcgagccaaccgggcaagccgccgaaagacgagctg